(19) United States
(12) Patent Application Publication
(10) Pub. No.: US 2016/0296555 A1
(43) Pub. Date: Oct. 13, 2016

(54) COMBINATION OF PYRIDOXINE, FOLIC ACID AND MAGNESIUM IONS FOR TREATING CANCER

(71) Applicant: Ramiro Moises VERGARA CAMPILLO, Bogotá (CO)

(72) Inventor: Ramiro Moises VERGARA CAMPILLO, Bogotá (CO)

(21) Appl. No.: 15/100,642

(22) PCT Filed: Dec. 20, 2013
(86) PCT No.: PCT/IB2013/002861

§ 371 Date: Jun. 1, 2016

(51) Int. Cl. A61K 33/06; A61K 31/4415; A61K 9/20; A61K 9/00; A61K 9/08; A61K 33/10; A61K 31/519

(52) U.S. Cl. CPC A61K 33/06; A61K 33/10; A61K 31/4415; A61K 31/519; A61K 9/0019; A61K 9/08; A61K 9/20

(57) ABSTRACT

The present invention relates to a new composition, made up of pyridoxine, folic acid and magnesium ions in chloride salt form for the manufacture of a medicinal product for treating cancer with the right combination of these active ingredients, which cover the 360 degree spectrum in the brain, as regards regulation of the synthesis of the four main neurotransmitter in the brain, working by virtue of the combination with the required proportions of the substances proposed herein, restoring normal tissue behaviour at the site, eliminating malignant and benign tumors without the need to use destructive methods such as chemotherapy, radiation therapy and organ harvesting. Said composition may be administered intravenously or in the form of tablets or syrup.

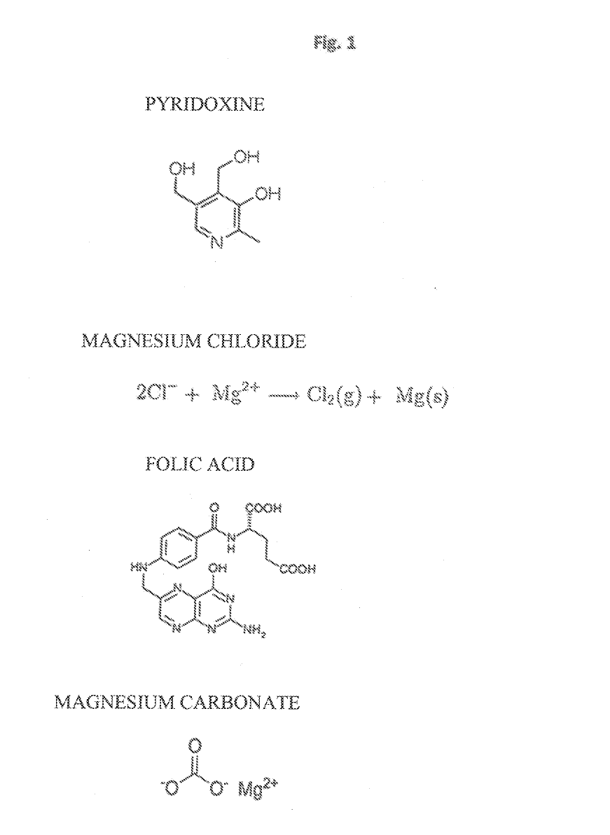

### SUMMARY

This hereby invention is related to a now composition, conformed by PYRIDOXINE, folic acid and magnesium IONS IN A CHLORIDE salt form, for the elaboration of a medication for the cancer treatment with the exact combination of such active ingredients, which cover 360° spectrum of the brain, in the regulation of the synthesis of the 4 main neurotransmitters of the brain, intervening with the combination of the necessary proportions with the substances hereby propose, which will restore the normal behavior of tissues up to point, which malign and benign tumors disappears without the necessary to use distractive procedures such as; chemotherapy, radiotherapy and Oregon removal. The administration of this compound (composition) is intravenously syrup and tablets

### DESCRIPTION OF THE INVENTION

### State-of-the-Art

Up until this time medical science approaches the pathology of malign and benign to tumors based of the great principles, which are, external NOXA and the localization of in the illness. THE PRINCIPLE OF THE EXTERNAL NOXA stipulates that all illness of the body is generated by an external agent, which penetrates the organism by any type of solution of continuity within the skin or mucus tissue which cover the same, which becomes that gates of entry for the PATHOGEN AGENT, which once within produces they illness due to its own pathogenic property, without considering the condition of host.

The principle of the location of illness, some stains that illnesses compromise only one organ or system of those which comprise the body and it focuses is activity upon discovering this location and combating the pathology within the discovered location, believing that by eliminating the ill organ they will achieve restoring the good health care state.

Current medical science adopts a bayou and attitude before pathology and therefore proposes combating illnesses through destructive procedures, with which it attacks the ill organ. They used 3 procedures which are surgery, radiotherapy and chemotherapy. With surgery the intent removing the ill organ or a great portion of the St. which includes the tumors and great neighboring mass tissue. For radiotherapy they use radioactive wave generating sources with which they intend to destroy the tumors, but it is inevitable to penetrate other tissues which are healthy and are also destroyed at the same time. For chemotherapy date is typical substances which are highly toxic for the cell membrane whit which they intend destroying the tumor but it is inevitable that such substances once in blood circulation will attack healthy organs and tissue. The active components, to which this invention refers to, pyridoxine, folic acid and magnesium ions on a chloride salt, are not included jointly, but separately as we can see him patents number WO 200-804-8085 A1, WO 200-109-5887 A1 AND WO 201-104-1920 A8 Which are presented to follow, the following medication which solves the problem of cancer without producing collateral effects

### OBJECTIVES

One of the most important objectives of our new medication is that within the treatment of cancer after restoring normal behaviors of the tissues tumors will disappear, and it does not only sold the present to more, but also impedes and cures the metastasis which made have appeared, and impedes the appearance of new tumors and does not have secondary effects

Another in port and objective is that for the patient does not have any negative or undesirable effect, for the contrary it helps increasing vitality improving its quality of life from the same time of the treatment commencement.

Another objective is that for the pyridoxine, folic acid and magnesium ions on a chloride salt and/or carbonate, by being associated and simultaneously administered on exact proportions, they covered the normal functional spectrum of 360° on the regulation of the synthesis of 4 main neurotransmitters of the brain.

### DETAILED DESCRIPTION OF THE INVENTION

The hereby invention refers to a new composition of a medication for the cure of malign and benign tumors, and is come posed of a group of dome tenses for which their combination does not exist on the worlds pharmaceutical industry. Pyridoxine, folic acid and magnesium ions these substances which are constituted have been utilized separately, on dietary supplements, but up until now in combination they have never been utilized as medications.

This medications is formed by the combination of active ingredients, composed by the combination of pyridoxine, folic acid and magnesium ions containing 2 vitamins and one trace elements, which traditionally have been prescribed as aids in the treatment of illnesses in which the general state and the vitality of the patient are at risk, but they are not indicated mandatorily, but only after the treating medic reveals or receives a “hunch”, under its own discretion, they were added to basic and main treatments, which are generally those which are relied upon the expect that results. There are healthcare professionals which do not advise their use due to considering them a loss of resources.

This composition is the result, of our research work with the new methodology of research, which house then possible to discover that the illness has an especial mechanism of neuro-physiological nature and the physiological causality; this discovery carried out and up roads for a new therapeutic invention. The methodology employed has been denominated new applied clinical scientific investigation (NICCA) and consist on the scientific clinical added observation and potentiated whit the information which arises from psychoanalysis, neuroscience and the neurolinguistics programming.

With this new tool it is possible to see how the pathology behavior of organism is at the same as the normal one, but if it was observed by a magnifying glass; magnified and at the same time is was appreciated as how they invention is for the brain in order to govern the peripheral tissues and organs on its normal state; and how the brain has the necessary hardware and soft or in order to induce the pathological state and also, to after its therapeutically correct intervention, reverse it back to normality

The NICCA has allowed the signing therapeutic medication interventions, which are capable to cure illnesses which up until now had been considered as incurable, such as this composition which covers the 360° spectrum of the brain, within the regulation of synthesis of the 4 main neurotransmitter of the brain, based on the metabolism of Nero transmitter, which is the main metabolic pathway for the synthesis of the Gaba based on glutamic acid, which is carried out by the action of glutamic acid decarboxylase (GAD), for which it requires as a CO-FERMENT, PYRIDOXAL PHOSPHATE, PYRIDOXINE SALTS, plus phosphoric acid and also magnesium ions are required (MG++)

That same sense the synthesis for glycine which takes place from serine, due to the action of the enzyme serine-hydroxyl-methyl-transferase (SHMT), also requires pyridoxal phosphate as co-fermenting agent, as well as folic acid and magnesium ions (MG++)

The synthesis of glutamine, glutamine acid and Gaba, are performed through intimate cooperation between the new run and the cell at the glia, in such a way that between both they constitute Gaba shunt. Within this biochemical process intervened, on one side, the Gaba-transaminase enzyme (Gaba-T) and the Succinic Seminaldehyde Dehydrogenase (SSADH), which only works within the mitochondria an on the other side, Glutaminase or Glutaminic Acid Decarboxilase (GAD) and the Glutamine Syntetase (Glu-Synth), which are cytoplasmic enzymes. On the other hand the GAD only exists in the neurone and the Glu-Synt only at the glia. Here you can appreciate the important complementation of both cells, for the metabolism of the 2 neurotransmitters which are the most abundant within the central nervous system “CNS”.

The released Gaba within the synapse gap, is taken by the GabaT, in order to form semialdehyde succinic, which passes to succinic acid and once more enters the Krebs' cycle. The Glia at the same time forms glutamic acid, which is the Gaba precursor, based on the Alpha-oxoglutaric acid, by action of the aspartato aminotransferase, but this metabolic pathway is interrupted here and does not reach the formation of Gaba, since the cell of the Glia lacks GAD, on the other hand it has glutamine synthetase, which acts upon the glutamic acid to form glutamine which passes to the neuron, where it serves to for glutamic acid by action of the Glutaminase. The source of glutamic acid within the neuron is glucose, from which the Alpha oxoglutaric acid is derived, which transaminases by the aspartato aminotransferase, to form glutamic acid, this is transformer in to Gaba by the decarboxylase of the glutamic acid (GAD), the degradation of the Gaba is performed by the Gaba transaminase to form semi aldehydesuccinic. But this reaction can only take place, if the amine group acceptor is once more alpha oxoglutaric acid, which can create a new molecule of glutamic acid, which is a precursor of the Gaba.

This reveals the importance of the Gaba for the nervous system, since it grants this biochemical protection to set molecule, in order to ensure a permanent provision of Gaba to the neuron, the prior means that gaba Molecule can only be destroyed metabolically, if at the same time there is a formation of a molecule of fits precursor, in order to take its place.

Intervening with a combination of the necessary portions with the substances hereby proposed, with ECOSAN which restores the normal behavior of tissues deemed capable and efficient for the treatment of tumors, whether benign or malign, since the NICCA has promised discovering the efficient causality and the mechanism of both classes of tumors. Without the necessity to use surgery, radiotherapy and chemotherapy.

By the methods to treat tumors they comprised this 3 procedures which also generate some secondary if acts which are destructive this. With surgery patients are extensively mutilated and have their functions reduced; with radiotherapy there are damages produced has ulcerations which never healed; and chemotherapy produces generalized damage within the nervous system, tissues which generate blood cells, among others.

Proposed treatment—UTILIZING NICCA as the research method, we have been able to establish that something most enter the organism in order to generate and illness, it does not pertain to any type of microscopic agent, but simply the information, which constitutes the unconscious mind which adds to the brain and expresses within the body the changes produced based on the information received by the organs of the senses which act as external agents of the brain.

It is pertinent to note that the mind his comprised of information which are similar to short movies which comprise it, which are grouped by sectors of the information content related which acquires own autonomy, in the such a way that they become in the pendants from well, react independently before new content of information with enters the organs of the senses, reason for which they produce effects which may be destructive.

NICCA has also allowed establishing that if symptoms and clinical signs are perceived by the patient in a limited part of its body, this location is truly the place selected by the unconscious mind for express and its message, but the action of the information takes place within the center, in the brain in which the information content is related, between each other. This means that when symptom appears, the decision to send this massage has been unanimous by all instances of the system, which selects and location in order to disclose something pertaining to the information which the illness generates.

Furthermore, the NICCA has allowed establishing that both the unconscious mind as well as the brain have the property to be supplementary versatile, in other words, that they can perform one thinking as well the opposite, for which they require therapeutic intervention based on the principles as stipulated on this hereby document, as well as it was suggested by ECOSAN.

Due to the aforementioned the treatment which is per post on this hereby application of a request for patent of invention, is intended to increase the vitality of the host, in other words, the person which presents the illness, which directed to DE_PROGRAMING THE DESTRUCTIVE IMPULSES which have arisen from the same, but without generating any type of undesirable negative effect, also doesn't have any type of contradiction nor is it incompatible with other medication chemical substances.

This “ECOSAN” composition is constituted by 3 active ingredients which are:
A. PYRIDOXINE
B: FOLIC ACID
C: MAGNESIUM IONS; IN CARBONATE OR CHLORIDE SALT FORM

Constituting a composition, which can be administered orally, but for the effective treatment of tumors is required to be administered in a parenteral manned or intravenously, the concentration required which also constitute novelty, since current diet information informs that a normal adult requires 3 mg of pyridoxine per day, which are included in food, in case of tumors a dose of one gram IV is required, which according to the state of the patient may require more than one dose every 24 hours. For folic acid 100 micrograms per day are recommended for adult, which a included within food, but for the treatment of tumors 20 milligrams doses are required intravenously, which according to the state of the patient may require over one single dose every 24 hours. For the magnesium ion, 20 milligrams are recommended par day for adult, which are included within food, for the treatment of tumors a dose of 500 milligrams is required intravenously, which according to the state of the patient may require over one single dose every 24 hours.

1. Medication for the treatment of cancer characterize because it restores the normal behavior of tissues, for combinations of various proportions of PYRIDOXINE, folic acid and magnesium IONS AS CHLORIDE OR CARBONATE.

2. Medication for cancer treatment according to claim 1 characterized because it restores the normal behavior of tissues, due to the composition of the mixture of; PIRIDOXINE 66% folic acid 9% and magnesium chloride 25%.

3. Medication for cancer treatment according to claim 1 characterized because it restores the normal behavior of tissues, due to the composition of the mixture of; PIRIDOXINE 65% folic acid 8% and magnesium chloride 27%.

4. Medication for cancer treatment according to claim 1 characterized because it restores the normal behavior of tissues, due to the composition of the mixture of; PIRIDOXINE 65% folic acid 9% and magnesium chloride 26%.

5. Medication for cancer treatment according to claim 1 characterized because it restores the normal behavior of tissues, due to the composition of the mixture of; PIRIDOXINE 65% folic acid 10% and magnesium chloride 25%.

6. Medication for cancer treatment according to claim 1 characterized because it restores the normal behavior of tissues, due to the composition of the mixture of; PIRIDOXINE 64% folic acid 9% and magnesium chloride 27%.

7. Medication for cancer treatment according to claim 1 characterized because it restores the normal behavior of tissues, due to the composition of the mixture of; PIRIDOXINE 63% folic acid 8% and magnesium chloride 29%.

8. Medication for cancer treatment according to claim 1 characterized because it restores the normal behavior of tissues, due to the composition of the mixture of; PIRIDOXINE 62% folic acid 7% and magnesium chloride 31%.

9. Medication for cancer treatment according to claim 1 characterized because it restores the normal behavior of tissues, due to the composition of the mixture of; PIRIDOXINE 61% folic acid 6% and magnesium chloride 33%.

10. Medication for cancer treatment according to claim 1 characterized because it restores the normal behavior of tissues, due to the composition of the mixture of; PIRIDOXINE 60% folic acid 5% and magnesium chloride 35%.

11. Medication for cancer treatment according to claim 1 characterized because it restores the normal behavior of tissues, due to the composition of the mixture of; PIRIDOXINE 90% folic acid 2% and magnesium chloride 8%.

12. Medication for cancer treatment according to claim 1 characterized because it restores the normal behavior of tissues, due to the composition of the mixture of; PIRIDOXINE 85% folic acid 1% and magnesium chloride 14%.

13. Medication for the treatment of cancer according to claim 1 characterized because it restores the normal behavior of tissues, due to the composition of the mixtures on the prior percentages, for the fabrication of injectable items on an intravenous route.

14. Medication for the treatment of cancer according to claim 1 characterized because it restores the normal behavior of tissues, due to the composition of the mixtures by the prior percentages, for the manufacturing of syrups.

15. Medication for the treatment of cancer according to claim 1 characterized because it restores the normal behavior of tissues, due to the composition of the mixtures by the prior percentages, for the manufacturing of tablets, replacing the magnesium chloride, by magnesium carbonate whit the same percentages.
